(19) United States
(12) Patent Application Publication
(10) Pub. No.: US 2013/0142781 A1
(43) Pub. Date: Jun. 6, 2013

(54) PEG BASED HYDROGEL FOR PERIPHERAL NERVE INJURY APPLICATIONS AND COMPOSITIONS AND METHOD OF USE OF SYNTHETIC HYDROGEL SEALANTS

(71) Applicant: InVivo Therapeutics Corporation, Cambridge, MA (US)

(72) Inventors: Robert S. Langer, Newton, MA (US); Timothy O'Shea, Cambridge, MA (US); Jonathan R. Slotkin, Potomac, MD (US); Francis M. Reynolds, Lafayette Hill, PA (US); Alex Aimetti, Waltham, MA (US); Brian Hess, Charlestown, MA (US)

(73) Assignee: INVIVO THERAPEUTICS CORPORATION, Cambridge, MA (US)

(21) Appl. No.: 13/692,458

(22) Filed: Dec. 3, 2012

Related U.S. Application Data
(60) Provisional application No. 61/566,564, filed on Dec. 2, 2011.
(60) Provisional application No. 61/726,290, filed on Nov. 14, 2012.

(51) Int. Cl. A61K 47/20; A61K 31/795; A61L 24/04

(52) U.S. Cl. CPC A61K 47/20; A61L 24/046; A61K 31/795
USPC 424/130.1; 514/785; 514/17.7; 514/44 R; 514/169; 424/94.1; 514/626; 514/179; 514/282; 424/94.5; 514/8.4; 514/329; 514/550

(57) ABSTRACT

Hydrogels that may be used for treating peripheral nerves and related methods are provided. Synthetic hydrogel sealants, methods of forming synthetic hydrogel sealants, and the use of synthetic hydrogel sealants are provided.

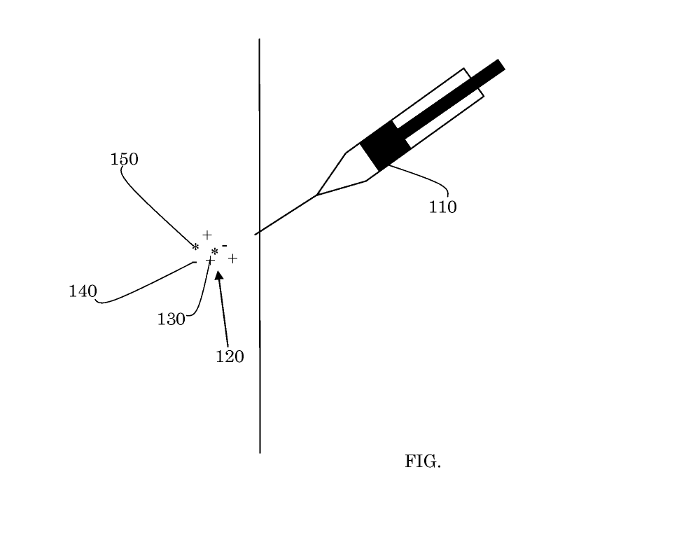

### CROSS REFERENCE TO RELATED APPLICATIONS

This application claims the benefit of U.S. provisional application No. 61/566,564, filed Dec. 2, 2011, and U.S. provisional application No. 61/726,290, filed Nov. 14, 2012, both of which are incorporated herein by reference as if fully set forth.

### FIELD OF INVENTION

The disclosure relates to hydrogels for treatment of peripheral nerve injury methods related thereto, synthetic hydrogel sealants, methods of forming synthetic hydrogel sealants, and the use of synthetic hydrogel sealants.

### SUMMARY

In an aspect, the invention relates to a method of treating a peripheral nerve. The method includes administering a PEG based hydrogel to patient in need thereof. The PEG based hydrogel comprises an overall polymer weight concentration of less than or equal to 50% at the time of curing. Administering includes applying the PEG based hydrogel to a site at or near the peripheral nerve.

In an aspect, the invention relates to a composition comprising a PEG based hydrogel with an overall polymer weight concentration of less than or equal to 50% at the time of curing.

In an aspect, the invention relates to a method of sealing tissue. The method includes mixing a first component and a second component to form a crosslinked hydrogel. The first component has a degree of functionality greater than or equal to three and is selected from the group consisting of a branched monomer, a multifunctional monomer, a branched polymer and a multifunctional polymer. The second component has a degree of functionality greater than or equal to two and is selected from the group consisting of a branched monomer, a multifunctional monomer, a branched polymer and a multifunctional polymer. The method also includes administering the first component and second component to tissue in situ prior to or during the step of mixing.

### BRIEF DESCRIPTION OF THE DRAWING

The following detailed description of the preferred embodiment of the present invention will be better understood when read in conjunction with the appended drawing. For the purpose of illustrating the invention, there is shown in the drawing an embodiment. It is understood, however, that the invention is not limited to the precise arrangements and instrumentalities shown. In the drawing:

The FIGURE illustrates a method of treating a peripheral nerve by administering to a patient the PEG based hydrogel.

### DETAILED DESCRIPTION OF THE PREFERRED EMBODIMENTS

The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C,” means any individual one of A, B or C as well as any combination thereof.

An embodiment provides a polyethylene glycol (PEG) based hydrogel that contains an overall polymer weight fraction at the time of curing of less than or equal to 50%. The term hydrogel means that an aqueous buffer or solution is present in the PEG based hydrogel. The PEG based hydrogel may be a vehicle for the controlled delivery of one or more therapeutic agents in peripheral nerve injury applications. The overall polymer weight fraction at the time of curing may be less than or equal to 50, 45, 40, 35, 30, 25, 20, or 15%. The overall polymer weight fraction is calculated by: (mass of polymer)/(mass of polymer+mass of aqueous buffer or solution). The overall polymer weight fraction at the time of curing may be a value within a range between and including any two integer weight fractions from 1-50%. In an embodiment, one or more therapeutic agent is provided in the PEG based hydrogel. The concentration of a therapeutic agent in the hydrogel may be selected to provide a dosage within the range of the clinically recommended dosage of the therapeutic agent. The therapeutic agent may be but is not limited to at least one of an anti-inflammatory drug, a surgical analgesia, an enzyme, or a growth factor. The one or more therapeutic agent may include the anti-inflammatory drug methylprednisolone. Non-limiting examples of the therapeutics that may be provided as the one or more therapeutic agent in the PEG based hydrogel are described in the US pre-grant application publication US 2010-0196481 (the publication of U.S. patent application Ser. No. 12/567,589), which is incorporated herein as if fully set forth.

The aqueous buffer or solution may be water. The aqueous buffer or solution may be a buffering medium. The buffering medium may have a basic pH. The buffering medium may have a pH in a range from 7.0 to 10.0 or a value in a range between any two pH values selected from 7.0 to 10.0 in 0.1 increments. For example, the pH may be in a range between 8.7 and 9.1. The pH may be any specific pH value selected from 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10. The pH may be 7.0-7.6.

The one or more therapeutic agent may provided in the PEG based hydrogel at a concentration in the hydrogel of 0.01-120 mg in 0.1 ml-20 ml of hydrogel. The amount of therapeutic agent in 0.1 ml-20 ml of hydrogel may be a value in a range between and including any two integer values from 1-120 mg. The amount of therapeutic agent in 0.1 ml-20 ml of hydrogel may be 0.01-1 mg. The concentration of a therapeutic agent in the hydrogel may be 20-120 mg in 0.5 ml-20 ml of hydrogel. The amount of therapeutic agent in 0.5 ml-20 ml of hydrogel may be a value in a range between and including any two integer values from 20 to 120 mg. The concentration of a therapeutic agent in the hydrogel may be from 0.1 mg/ml up to 12 mg/ml. The concentration of methylprednisolone in the hydrogel may be but is not limited to 2-120 mg in 0.1 ml-20 ml of hydrogel, or 20-120 mg in 0.5 ml-20 ml of hydrogel.

The PEG based hydrogel may include hydrolytically labile functional groups along the polymeric backbone of the hydrogel. The hydrolytically labile functional groups may include but are not limited to one or more type selected from the group consisting of esters, amides, anhydrides, epoxides, carbamates and ureas. The functional groups within the polymeric backbone may render the PEG based hydrogel hydrolytically degradable.

An embodiment provides a method of treating a peripheral nerve by administering to a patient in need thereof the PEG based hydrogel. The patient may be human. The patient may be non-human. The patient may be in need of treatment of a peripheral nerve that is injured or that may be injured in the course of an activity or medical procedure. Administering may include applying the PEG based hydrogel to a site at or near the peripheral nerve. Applying the PEG based hydrogel may include applying precursors of the PEG based hydrogel at the site such that the hydrogel forms in situ. Precursors may be the polymers that link to form the hydrogel. Precursors may be the first component and the second component described below with respect to the method of sealing tissue by forming a crosslinked hydrogel in-situ. The precursors may be in an aqueous buffer or solution. Applying may include injecting the PEG based hydrogel to the site. Referring to the FIGURE, injecting the PEG based hydrogel to the site may include injecting precursors 130 and 140 of the PEG based hydrogel to the site 120 such that the hydrogel forms in situ. The injecting may be accomplished with a syringe 110. Additional agents 150 may also be injected. The additional agents may include at least one of one or more therapeutic agent, a biological epitope, or a specific crosslinker. Examples of biological epitopes and specific crosslinkers are provided below. The PEG based hydrogel may be one as described herein. Non-limiting examples of hydrogels that may be the PEG based hydrogel in a method of treating a peripheral nerve are described in the US pre-grant application publication US 2010-0196481 (the publication of U.S. patent application Ser. No. 12/567,589), which is incorporated herein as if fully set forth.

In an embodiment of the method of a treating peripheral nerve, the volume of hydrogel administered may be in range of 0.1 to 20 ml. The volume may be 3-13 ml. The volume may be any value selected from 0.1 to 20 ml in 0.1 ml increments. For example, the volume may be 1.1 ml. The volume may have a value in a range between and including any two values from 0.1 to 20 ml in 0.1 ml increments. For example, the volume may have a value in a range from 0.3 to 15.1 ml. The volume administered may depend on the individual characteristics of the patient, and the location in which the hydrogel is applied; e.g., cervical and thoracic interlaminar injections; cervical and thoracic transforaminal injections; lumbar interlaminar injections; lumbar transforaminal injections; or lumbar caudal injections. Data suggests that the cumulative release kinetics of the drug from the hydrogel is independent of concentration. The skilled artisan will thus understand from the nature of the site of injection, the amount of hydrogel to apply, and the amount of therapeutic agent to include in the hydrogel.

The PEG based hydrogel may include and/or be used in a method herein to deliver at least one of the following therapeutic agents:
one or more corticosteroids, including but not limited to methylprednisolone;
one or more anesthetics including but not limited to lidocaine, bupivacaine, ropivacaine, or chloroprocaine;
one or more analgesics including but not limited to morphine, fentanyl, sufentanil, and pethidine; or
one or more growth factors including but not limited to nerve growth factor (NGF), brain-derived neurotrophic factor (BDNF), neurotrophin-3 (NT-3), neurotrophin-4/5 (NT-4/5), glial cell line-derived neurotrophic factor (GDNF), neurturin (NTN), persephin, or artemin.
   The concentration of one of the above therapeutic agents in the hydrogel may be selected to provide a dosage within the range of the clinically recommended dosage of the therapeutic agent. The concentration of one of the above therapeutic agents in the hydrogel may be 0.01-120 mg in 0.1 ml-20 ml of hydrogel. The amount of the therapeutic agent in 0.1 ml-20 ml of hydrogel may be a value in a range between and including any two integer values from 1-120 mg. The amount of the therapeutic agent in 0.1 ml-20 ml of hydrogel may be 0.01-1 mg. The concentration of the therapeutic agent in the hydrogel may be 20-120 mg in 0.5 ml-20 ml of hydrogel. The amount of the therapeutic agent in 0.5 ml-20 ml of hydrogel may be a value in a range between and including any two integer values from 20 to 120 mg. The concentration of one of the above therapeutic agents in the hydrogel may be from 0.1 mg/ml up to 12 mg/ml.

A general purpose of the PEG based hydrogel is for use in a variety of neurosurgical and interventional pain management applications. Applications where the hydrogel or methods herein may be implemented include but are not limited to the following:
As part of an epidural steroid injection to mitigate pain, inflammation, or to reduce epidural fibrosis on the spinal dural sac and exiting neural elements;
As part of the procedure known as a selective nerve root block;
As part of the procedure known as a caudal injection;
As part of the procedure known as a facet block;
As part of the procedure known as a sacroiliac injection or block;
Treatment of carpal tunnel syndrome;
Treatment of lateral epicondylitis (Tennis elbow);
As a dural sealant for the spine, spinal cord, nerve roots, and surrounding structures;
As a dural sealant for cranial applications;
To deliver drugs, cells, and other substances to the spinal coverings, neural elements, and surrounding structures;
To deliver drugs, cells, and other substances to the intervertebral disc, disc annulus, end plates, and surrounding structures; and
To deliver drugs, cells, and other substances to the vertebral bones and articulating processes including the vertebral body, lamina, facet joint, joint capsule, pars interarticularis, neural foramen, transverse process, spinous process, and surrounding ligaments and other supporting structures.
   The method of treating a peripheral nerve for one of the above applications may include combining the hydrogel precursors in solution or suspension with any other agent for the application, and injecting the combination. The site of injecting would be known to the skilled artisan for each of the above applications. All therapeutics for an application could be incorporated within the gel precursor solution (prior to gelation) and become physically entrapped or covalently reacted within the hydrogel polymeric network thus only requiring one injection.

A non-limiting example of a PEG based hydrogel and its precursors that may be implemented in a method of treating a peripheral nerve is provided in Example 1, below. A method of sealing tissue is described below and a crosslinked hydrogel for the method. The crosslinked hydrogel there described and the methods of forming it may be provided as a PEG based hydrogel and related method as described above when the crosslinked hydrogel is a PEG based hydrogel.

An embodiment includes a technique for forming crosslinked hydrogels in-situ to be used as a tissue sealant, more specifically as a dural sealant. During various cranial or spine surgeries, achieving water-tight closure of the outer-most layer, the dura, is extremely difficult. Clinicians currently use staples, grafts, or inferior dural sealants in attempts to close the dura.

Embodiments include a method of sealing tissue by forming a crosslinked hydrogel in-situ. The methods may include mixing a first component and a second component to form the crosslinked hydrogel. The crosslinked hydrogel may be of infinite molecular weight. The crosslinked hydrogel may be the PEG based hydrogel described above.

The method of sealing tissue may include mixing a first component and a second component to form the crosslinked hydrogel. The first component may have a degree of functionality greater than or equal to three and may be selected from the group consisting of a branched monomer, a multifunctional monomer, a branched polymer and a multifunctional polymer. The second component may have a degree of functionality greater than or equal to two and be selected from the group consisting of a branched monomer, a multifunctional monomer, a branched polymer and a multifunctional polymer. The method may also include administering the first component and second component to tissue in situ prior to or during the step of mixing.

The tissue may be tissue damaged in an accident or surgery. The tissue may be dural tissue. The tissue may be cut, ruptured, or otherwise open and the step of administering may include applying the first component and the second component to at least one of on, in, or near the region of tissue cut, ruptured, or otherwise open. The volume of composition including the first component and the second component administered may be selected to suit the size of the region. The volume may be 3-13 ml. The volume may be in range of 0.1 to 20 ml. The volume may be any value selected from 0.1 to 20 ml in 0.1 ml increments. For example, the volume may be 1.1 ml. The volume may have a value in a range between and including any two values from 0.1 to 20 ml in 0.1 ml increments. For example, the volume may have a value in a range from 0.3 to 15.1 ml.

The first component may have an electrophilic functional group and the second component may have a nucleophilic functional group. The first component may have a nucleophilic functional group and the second component may have an electrophilic functional group.

Mixing may include adding the first component and the second component in stoichiometric equivalencies relative to functional groups. Mixing may include adding the first component and the second component in a buffering medium. The buffering medium may be aqueous. The buffering medium may have a basic pH. The buffering medium may have a pH in a range from 8.5 to 9.5 or a value in a range between any two pH values selected from 8.5 to 9.5 in 0.1 increments. For example, the pH may be in a range between 8.7 and 9.1. The pH may be any specific pH value selected from 8.5, 8.6, 8.7, 8.8, 8.9, 9.0, 9.1, 9.2, 9.3, 9.4, or 9.4. The buffering medium may have a pH in a range from 7.0 to 10.0 or a value in a range between any two pH values selected from 7.0 to 10.0 in 0.1 increments. The pH may be any specific pH value selected from 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10. The pH may be 7.0-7.6.

The crosslinked hydrogel may have a polymer weight percent between and including 10 to 30 percent. The crosslinked hydrogel polymer weight percent may be any value between 10 and 30 percent. The crosslinked hydrogel weight percent may have a value in a range between and including any two integer percents selected from 10 to 30. For example, the weight percent may have a value between and including the range from 12 to 21 percent.

The method may include incorporating at least one type of biological epitope in the crosslinked hydrogel. The biological epitope may be a peptide, a protein, an antibody or an aptamer. The peptide may be but is not limited to RGD, IKVAV and YIGSR. RGD or IKVAV peptides can be used to promote cellular attachment, direct cell differentiation, and promote regeneration. The protein may be a nerve growth factor. The nerve growth factor may be NT-3. The antibody may be an NT-3 antibody. The peptide may be NQEQVSPK(βA)FAKLAARLYRKA-NH2. One or both of the NT-3 antibody or NQEQVSPK(βA)FAKLAARLYRKA-NH2 may be included with NT-3 and may control the release of NT-3 from the gel. The antibody or aptamer may be Anti-Nogo antibodies or aptamer antagonists to the Nogo-66 (NgR) receptor, which could be included to promote axon elongation. Incorporating a biological epitope may include adding the biological epitope to at least one of the first component or the second component prior to the step of mixing. Incorporating a biological epitope may include adding the biological epitope during mixing. Incorporating a biological epitope may include applying the biological epitope in situ at a stage of at least one of before, during, or after the step of administering.

The crosslinked hydrogel may be a poly(ethylene glycol) (PEG)-based hydrogel formed via a base-catalyzed Michael-type reaction after the step of mixing. Poly(ethylene glycol) may be utilized as a biocompatible base polymer. The crosslinked hydrogel may be an alternative polymeric species. The step of mixing may include mixing PEG-diacrylate (Mn˜700 g mol⁻¹) and ethoxylated-trimethylolpropan tri(3-mercaptopropionate) (ETTMP) (Mn˜1300 g mol⁻¹) in stoichiometric equivalence relative to acrylate (electrophile) and thiol (nucleophile) concentrations. Non-limiting examples of crosslinked hydrogels that may be formed and implemented herein can be found in the US pre-grant application publication US 2010-0196481 (the publication of U.S. patent application Ser. No. 12/567,589, filed Sep. 25, 2009), which is incorporated herein by reference as if fully set forth.

The kinetics of the reaction may be dependent on the pH of the buffering medium. For tissue sealant applications where fast cure rates are desired, the gel may be fabricated in pH 8.5-9.5 medium since the Michael-type reaction is base-catalyzed. The crosslinked hydrogel may exhibit no swell or syneresis (shrinkage) at a polymer weight percent ranging from 10% polymer to 30% polymer. This is desirable for sealant applications in confined spaces such as the spine.

An embodiment of the method of sealing tissue may include applying different crosslinked hydrogels having different viscosities, which may be independently tuned to achieve enhanced adhesiveness to various tissues. For example, the method may include applying components forming a low viscosity gel solution in situ followed by applying components forming a higher viscosity. As a non-limiting example a low viscosity gel solution may be between 1-5 cPs and high viscosity gel solution may be between 5-100 cPs. The method may include applying a low viscosity gel solution than can slightly penetrate within the tissue. At that point the preferred gel formulation may be delivered that could further react with the initial layer forming a robust interpenetrating network where strong mechanical adhesive forces were present.

Embodiments of the crosslinked hydrogel may accommodate various biological epitopes that can be covalently incorporated within the material. For example, if desired, various peptides such as RGD, IKVAV, YIGSR could be used in another combination to achieved a desired effect included but not limited to cell proliferation, differentiation, attachment, or promote migration. Alternatively, one could use peptides as a means to control or enhance electrostatic interactions with the tissue to be adhered to. The biological epitope may be a peptide, a protein, an antibody or an aptamer. The peptide may be but is not limited to RGD, IKVAV and YIGSR. RGD or IKVAV peptides can be used to promote cellular attachment, direct cell differentiation, and promote regeneration. The protein may be a nerve growth factor. The nerve growth factor may be NT-3. The antibody may be an NT-3 antibody. The peptide may be NQEQVSPK(βA)FAKLAARLYRKA-NH2. One or both of the NT-3 antibody or NQEQVSPK(βA)FAKLAARLYRKA-NH2 may be included with NT-3 and may control the release of NT-3 from the gel. The antibody or aptamer may be Anti-Nogo antibodies or aptamer antagonists to the Nogo-66 (NgR) receptor, which could be included to promote axon elongation. Incorporating a biological epitope may include adding the biological epitope to at least one of the first component or the second component prior to the step of mixing. Incorporating a biological epitope may include adding the biological epitope during mixing. Incorporating a biological epitope may include applying the biological epitope in situ at a stage of at least one of before, during, or after the step of administering. To conjugate a peptide within a gel one could exploit the sulfhydryl group that natively exists on cysteine amino acids as a convenient way to react the peptide within the gel. Alternatively, one could use conjugation techniques to modify the peptide with vinyl functionality including acrylates. Peptides may be incorporated in the method in a similar fashion as the biological epitope, described above.

A crosslinked hydrogel herein may include enzymatically labile functional groups or moieties incorporated within the polymeric backbone of the hydrogel (either as a result of the chemical reaction, or previously designed within the polymer chain). For example, functional groups such as esters are not only degraded by simple hydrolysis, but are also degraded by esterases. A specific esterase is lipase. The chemical reaction to form the hydrogel may result in an ester group, or the group could previously be designed within the polymer precursor. Enzymes bind reversibly with their substrates and convert the substrate to product. Substrates can be proteins, proteoglycans, sugars, peptides, etc. A functional group for an enzyme or a substrate could be included in a crosslinked hydrogel as an enzymatically labile functional group or moiety, respectively. The substrate may be linked to the hydrogel. The enzymatically labile functional group or substrate may crosslink the hydrogel. This would render the gel degradable when the enzyme is up-regulated. Specific enzymes that the enzymatically labile functional group or substrate could be designed for may be pro-inflammatory enzymes (human neutrophil elastase, cyclooxygenase-2, heme oxygenases, etc.) or matrix-metalloproteases (MMPs). By inclusion of such a functional group or substrate, upregulation of these enzymes may lead to degradation of the hydrogel.

A crosslinked hydrogel herein may include a specific crosslinker, which may be used to covalently bond the hydrogel material to the surrounding tissue. A method of sealing tissue may include incorporating a specific crosslinker. Various hetrobifunctional linkers exist that may react within the gel as well as react to available functional groups (amines, carboxylic acids, etc.) within extracellular matrix molecules within the tissue such as collagen. For example, succinimidyl-([N-maleimidopropionamido]-ethyleneglycol) ester is a crosslinker that may be used to react within the gel (maleimide) as well as bond with ε or α-amino groups on collagen which is a critical component in most tissues. Any of the above may be the specific crosslinker. Incorporating a specific crosslinker may include adding the specific crosslinker to at least one of the first component or the second component prior to the step of mixing. Incorporating a specific crosslinker may include adding the specific crosslinker during mixing. Incorporating a specific crosslinker may include applying the specific crosslinker in situ at a stage of at least one of before, during, or after the step of administering.

A crosslinked hydrogel herein may include one or more therapeutic agent. The one or more therapeutic agent may be as described above with respect to the PEG based hydrogel. The concentration of a therapeutic agent in the crosslinked hydrogel may be as described above with respect to the PEG based hydrogel. A method of sealing tissue may include a crosslinked hydrogel with one or more therapeutic agent. The method may include encapsulating the one or more therapeutic agent at the time of application, which may aide in the healing process. The one or more therapeutic agent may be regenerative, anti-inflammatory, or analgesic in nature. Encapsulating one or more therapeutic agent may include adding the one or more therapeutic agent to at least one of the first component or the second component prior to the step of mixing. Encapsulating one or more therapeutic agent may include adding the one or more therapeutic agent during mixing. Encapsulating one or more therapeutic agent may include applying the one or more therapeutic agent in situ at a stage of least one of before, during, or after the step of administering.

The one or more therapeutic agent in a crosslinked hydrogel, or in a method of sealing tissue herein may include at least one of a steroid, methylprednisolone, dexamethasone, prodrugs of methylprednisolone, prodrugs of dexamethasone, inhibitors of NOS or NO production, an antioxidant or antioxidants, spin traps, peroxynitrite scavengers, minocycline, vitamin C, vitamin E, tempol (4-hydroxy-2,2,6,6-tetramethylpiperidine-1-oxyl), uric acid, and MnTBAP, or a pharmaceutically acceptable salt of any of the foregoing. Non-limiting examples of the therapeutics that may be encapsulated are described in the US pre-grant application publication US 2010-0196481 (the publication of U.S. patent application Ser. No. 12/567,589), which is incorporated herein as if fully set forth.

Embodiments include the product or formulation of any method or technique above.

Any of the above products or formulations can be delivered in a double-barreled syringe. The step of at least one of mixing or administering may include dispensing one or more substance in the method with a syringe in situ, and the syringe may be but is not limited to a double barreled syringe.

The crosslinked hydrogel may be designed to not swell when cured and equilibrated in-vivo. In some cases, the crosslinked hydrogel exhibits syneresis (shrinks) which may be desirable to provide mechanical stresses to aide in wound closure. It may be undesirable to experience swelling >15% (volume) which puts unwanted stress on local nerve roots causing undesirable pain and other complications.

In some embodiments, the technology herein can be prepared in less than 2 minutes and engineered to cure at any rate above 3 seconds to meet a surgeon need.

### Embodiment List

The following list includes particular embodiments. The list, however, is not limiting and does not exclude alternate embodiments otherwise described or as would be appreciated by one of ordinary skill in the art.

1. A method of treating a peripheral nerve comprising: administering a PEG based hydrogel to patient in need thereof, the PEG based hydrogel comprising an overall polymer weight concentration of less than or equal to 50% at the time of curing, wherein administering includes applying the PEG based hydrogel to a site at or near the peripheral nerve.

2. The method of embodiment 1, wherein applying the PEG based hydrogel includes applying a composition including precursors of the PEG based hydrogel to the site.

3. The method of embodiment 2, wherein applying the composition includes in injecting the composition at the site.

4. The method of any one or more of embodiments 2-3, wherein the composition includes an aqueous buffer or solution.

5. The method of any one or more of embodiments 2-3, wherein the composition includes at least on additional agent selected from the group consisting of one of one or more therapeutic agent, a biological epitope, and a specific crosslinker.

6. The method of any one or more of the preceding embodiments, wherein the PEG based hydrogel is formed via a step growth, base-catalyzed reaction between a donor and an acceptor, the donor having a nucleophilic functional group and the acceptor having an electrophilic functional group, wherein the precursors include the donor and the acceptor.

7. The method of embodiment 6, wherein the nucleophilic functional group is a thiol and the electrophilic functional group is an acrylate and together the thiol and the acrylate form a thioether.

8. The method of any one or more of embodiments 6-7, wherein the donor is a trifunctional thiol polymer and the acceptor is a bifunctional acrylate polymer.

9. The method of any one or more of embodiments 6-8, wherein the donor is ethoxylated trimethylolpropane tri-3-mercaptopropionate and the acceptor is poly(ethylene glycol)diacrylate.

10. The method of any one or more of embodiments 6-9, wherein the PEG based hydrogel comprises at least one type of hydrolytically labile functional group selected from the group consisting of esters, amides, anhydrides, epoxides, carbamates and ureas.

11. The method of any one or more of the preceding embodiments, wherein the PEG based hydrogel includes a biological epitope selected from the group consisting of a peptide, a protein, an antibody and an aptamer.

12. The method of any one or more of the preceding embodiments, wherein the composition includes a biological epitope selected from the group consisting of a peptide, a protein, an antibody and an aptamer.

13. The method of any one or more of embodiments 11 or 12, wherein the biological epitope is selected from the group consisting of RGD and IKVAV.

14. The method of any one or more of the preceding embodiments, wherein the PEG based hydrogel includes enzymatically labile functional groups or substrates.

15. The method of any one or more of the preceding embodiments, wherein the PEG based hydrogel further comprises at least one therapeutic agent.

16. The method of any one or more of the preceding embodiments, wherein the composition further comprises at least one therapeutic agent.

17. The method of any one or more of embodiments 15-16, wherein the at least one therapeutic agent has a concentration of 0.01 mg to 120 mg in 0.1 ml to 20 ml of the PEG based hydrogel.

18. The method of any one or more of embodiments 15-17, wherein the at least one therapeutic agent is selected from the group consisting of an anti-inflammatory drug, a steroid, a surgical analgesia, an enzyme, and a growth factor.

19. The method of any one or more of embodiments 15-18, wherein the at least one therapeutic agent includes at least one of corticosteroid, methylprednisolone, an anesthetic, lidocaine, bupivacaine, ropivacaine, chloroprocaine, an analgesic, morphine, fentanyl, sufentanil, pethidine, an enzyme, chrondrotinase ABC, a growth factor, neurotrophin-3, nerve growth factor, or brain-derive neurotrophic factor.

20. The method of any one or more of the preceding embodiments, wherein administering includes delivering 0.1 ml to 20 ml of the PEG based hydrogel.

21. The method of any one or more of the preceding embodiments, wherein administering includes delivering 0.1 ml to 20 ml of the composition.

22. The method of any one or more of the preceding embodiments, wherein administering includes delivering the PEG based hydrogel as at least part of one of the group consisting of an epidural steroid injection, a selective nerve root block procedure, a caudal injection procedure, a facet block procedure, a sacroiliac injection or block procedure, a treatment of carpal tunnel syndrome, a treatment of lateral epicondylitis, a dural sealant, a substance delivery system, and treating an articulating process.

23. The method of any one or more of the preceding embodiments, wherein administering includes delivering the composition as at least part of one of the group consisting of an epidural steroid injection, a selective nerve root block procedure, a caudal injection procedure, a facet block procedure, a sacroiliac injection or block procedure, a treatment of carpal tunnel syndrome, a treatment of lateral epicondylitis, a dural sealant, a substance delivery system, and treating an articulating process.

24. The method of any one or more of the preceding embodiments, wherein administering includes delivering the PEG based hydrogel by a cervical interlaminar injection, a thoracic interlaminar injection, a lumbar interlaminar injection, a lumbar transforaminal injection and a lumbar caudal injection.

25. The method of any one or more of the preceding embodiments, wherein administering includes delivering the composition by a cervical interlaminar injection, a thoracic interlaminar injection, a lumbar interlaminar injection, a lumbar transforaminal injection and a lumbar caudal injection.

26. A composition comprising a PEG based hydrogel with an overall polymer weight concentration of less than or equal to 50% at the time of curing.

27. The composition of embodiment 26, wherein the PEG based hydrogel is formed via a step growth, base-catalyzed reaction between a donor and an acceptor, the donor having a nucleophilic functional group and the acceptor having an electrophilic functional group.

28. The composition of embodiment 26, wherein the PEG based hydrogel is formed between two poly(ethylene glycol) (PEG) macromers where one of the macromers has a degree of branching equal to or greater than 3 while the other PEG macromer has a degree of branching equal to or greater than 2.

29. The composition of any one or more of embodiments 27-28, wherein the donor is a trifunctional thiol polymer and the acceptor is a bifunctional polymer.

30. The composition of any one or more of embodiments 27 and 29, wherein the donor is ethoxylated trimethylolpropane tri-3-mercaptopropionate and the acceptor is poly(ethylene glycol)diacrylate.

31. The composition of any one or more of embodiments 26-30, wherein the PEG based hydrogel comprises at least one type of hydrolytically labile functional group selected from the group consisting of esters, amides, anhydrides, epoxides, carbamates and ureas.

32. The composition of any one or more of embodiments 26-31, wherein PEG based hydrogel includes a biological epitope is selected from the group consisting of peptides, proteins, antibodies and aptamers.

33. The composition of embodiment 32, wherein the biological epitope is selected from the group consisting of RGD and IKVAV.

34. The composition of any one or more of embodiments 26-33, wherein the PEG based hydrogel includes enzymatically labile functional groups or substrates.

35. The composition of any one or more of embodiments 26-34, wherein the PEG based hydrogel further comprises at least one therapeutic agent.

36. The composition of embodiment 35, wherein the at least one therapeutic agent has a concentration of 0.01 mg to 120 mg in 0.1 ml to 20 ml of crosslinked hydrogel.

37. The composition of any one or more of embodiments 35-36, wherein the at least one therapeutic agent is selected from the group consisting of an anti-inflammatory drug, a surgical analgesia, an enzyme and a growth factor.

38. The composition of any one or more of embodiments 35-37, wherein the at least one therapeutic agent includes at least one of corticosteroid, methylprednisolone, an anesthetic, lidocaine, bupivacaine, ropivacaine, chloroprocaine, an analgesic, morphine, fentanyl, sufentanil, pethidine, an enzyme, chrondrotinase ABC, a growth factor, neurotrophin-3, nerve growth factor, or brain-derive neurotrophic factor.

39. A method of sealing tissue comprising:
mixing a first component and a second component to form a crosslinked hydrogel, the first component having a degree of functionality greater than or equal to three and selected from the group consisting of a branched monomer, a multifunctional monomer, a branched polymer and a multifunctional polymer; the second component having a degree of functionality greater than or equal to two and selected from the group consisting of a branched monomer, a multifunctional monomer, a branched polymer and a multifunctional polymer; and
administering the first component and second component to tissue in situ prior to or during the step of mixing.

40. The method of embodiment 39, wherein one of the first component or the second component contains electrophilic functional groups and the other of the first component or the second component contains nucleophilic functional groups.

41. The method of any one or more of embodiments 39-40, wherein mixing includes adding the first component and the second component in stoichiometric equivalencies relative to functional groups.

42. The method of any one or more of embodiments 39-41, wherein mixing includes adding the first component and the second component in a buffering medium.

43. The method of embodiment 42, wherein the buffering medium has a pH of greater than seven.

44. The method of embodiment 42, wherein the buffering medium has a pH of 8.5 to 9.5.

45. The method of any one or more of embodiments 39-44, wherein the first component is PEG-diacrylate and the second component is ethoxylated-trimethylolpropan tri(3-mercaptopropionate).

46. The method of any one or more of embodiments 39-45, wherein mixing includes adding PEG-diacrylate and ethoxylated-trimethylolpropan tri(3-mercaptopropionate) in stoichiometric equivalence relative to acrylate and thiol concentrations.

47. The method of any one or more of embodiments 39-46, wherein the crosslinked hydrogel has a polymer weight percent of 10 to 30 percent.

48. The method of any one or more of embodiments 39-47 further comprising incorporating at least one biological epitope in the crosslinked hydrogel.

49. The method of embodiment 48, wherein the at least one biological epitope includes at least one peptide.

50. The method of embodiment 49, wherein the at least one peptide includes at least one of RGD, IKVAV or YIGSR.

51. The method of any one or more of embodiments 48-50, wherein incorporating includes conjugating one or more of the at least one peptide to the crosslinked hydrogel by reacting a sulfhydryl group thereon with a functional group on the crosslinked hydrogel.

52. The method of any one or more of embodiments 48-51, wherein incorporating includes modifying the peptide with vinyl functionality including acrylates using conjugation techniques.

53. The method of any one or more of embodiments 39-52 further comprising incorporating a crosslinker in the crosslinked hydrogel.

54. The method of embodiment 53, wherein the crosslinker is succinimidyl-([N-maleimidopropionamido]-ethyleneglycol) ester.

55. A composition comprising a product of mixing and or incorporating in any of embodiments 39-54.

56. A composition comprising a product produced by steps comprising:
mixing a first component and a second component to form a crosslinked hydrogel, the first component having a degree of functionality greater than or equal to three and selected from the group consisting of a branched monomer, a multifunctional monomer, a branched polymer and a multifunctional polymer; the second component having a degree of functionality greater than or equal to two and selected from the group consisting of a branched monomer, a multifunctional monomer, a branched polymer and a multifunctional polymer.

57. The composition of embodiment 56, wherein one of the first component or the second component contains electrophilic functional groups and the other of the first component or the second component contains nucleophilic functional groups.

58. The composition of any one or more of embodiments 56-57, wherein mixing includes adding the first component and the second component in stoichiometric equivalencies relative to functional groups.

59. The composition of any one or more of embodiments 56-58, wherein mixing includes adding the first component and the second component in a buffering medium.

60. The composition of embodiment 59, wherein the buffering medium has a pH of greater than seven.

61. The composition of embodiment 59, wherein the buffering medium has a pH of 8.5 to 9.5.

62. The composition of any one or more of embodiments 56-61, wherein the first component is PEG-diacrylate and the second component is ethoxylated-trimethylolpropan tri(3-mercaptopropionate).

63. The composition of any one or more of embodiments 56-62, wherein mixing includes adding PEG-diacrylate and ethoxylated-trimethylolpropan tri(3-mercaptopropionate) in stoichiometric equivalence relative to acrylate and thiol concentrations.

64. The composition of any one or more of embodiments 56-63, wherein the crosslinked hydrogel has a polymer weight percent of 10 to 30 percent.

65. The composition of any one or more of embodiments 56-64, wherein the steps further comprise incorporating at least one biological epitope in the crosslinked hydrogel.

66. The composition of embodiment 65, wherein the at least one biological epitope includes at least one peptide.

67. The composition of embodiment 66, wherein the at least one peptide includes at least one of RGD, IKVAV or YIGSR.

68. The composition of any one or more of embodiments 65-67, wherein incorporating includes conjugating one or more of the at least one peptide to the crosslinked hydrogel by reacting a sulfhydryl group thereon with a functional group on the crosslinked hydrogel.

69. The composition of any one or more of embodiments 65-68, wherein incorporating includes modifying the peptide with vinyl functionality including acrylates using conjugation techniques.

70. The composition of any one or more of embodiments 56-69, wherein the steps further comprise incorporating a crosslinker in the crosslinked hydrogel.

71. The composition of embodiment 70, wherein the crosslinker is succinimidyl([N-maleimidopropionamido]-ethyleneglycol) ester.

72. A method of treating a peripheral nerve comprising administering the composition of any one or more of embodiments 26-38 or 56-71 to a patient in need thereof, wherein administering includes applying the composition to a site at or near the peripheral nerve.

73 A method of sealing tissue comprising administering the composition of any one or more of embodiments 26-38 or 56-71 to tissue in situ prior to gelation.

74. A method of making a composition comprising the any method of forming a hydrogel described herein.

75. A method of making a composition comprising the steps other than administering or applying in any one or more of embodiments 1-25 and 39-54.

76. The method of any one or more of embodiments 39-55 or 73, wherein tissue is dural tissue damaged in an accident or surgery.

77. The method of embodiment 76, wherein, the dural tissue is cranial.

78. The method of embodiment 76, wherein the dural tissue is spinal.

Further embodiments herein may be formed by supplementing an embodiment with one or more element from any one or more other embodiment herein, and/or substituting one or more element from one embodiment with one or more element from one or more other embodiment herein.

### Examples

The following non-limiting examples are provided to illustrate particular embodiments. The embodiments throughout may be supplemented with one or more detail from one or more example below, and/or one or more element from an embodiment may be substituted with one or more detail from one or more example below.

### Example 1

### A Polyethylene Glycol (PEG) Based Hydrogel

A polyethylene glycol (PEG) based hydrogel that contains an overall polymer weight fraction at the time of curing of 15% has been developed as a vehicle for the controlled delivery of anti-inflammatory drugs and surgical analgesia in peripheral nerve injury applications. The PEG based hydrogel of this example transitions from an aqueous polymer sol mixture to a viscoelastic polymer network in situ via a base catalyzed thiol-ene Michael conjugate addition reaction involving two synthetic polymer constituents. The two polymers that comprise the system are: (1) the tri-functional thiol polymer, Ethoxylated Trimethylolpropane Tri-3-mercaptopropionate (ETTMP Thiocure® 1300, Bruno Bock; M_{w}˜1274 g/mol; viscosity 450 mPas; density=1.15 g/cm³); and (2) the bifunctional polymer, Poly(ethylene glycol)diacrylate (PEG-DA) [Sigma Aldrich, #455008; Mₙ˜675-725 g/mol; density=1.12 g/cm³]. The reaction takes place in an aqueous buffer under slightly basic conditions (pH 7.2-9.5; pH 7.6-9.5, depending on the application) and with approximately isotonic salt concentrations (solvent osmolality ranging between 260-300 mOsm/kg as measured by freezing point depression osmometry). In the current peripheral nerve application, a pH 7.2 isotonic phosphate buffered saline will be used.

Individual solutions of the two constituent polymers are prepared first and then mixed together at stoichiometric equivalent ratios to initiate the gelation reaction. Immediately prior to making the individual polymer solutions the bulk ETTMP 1300 and PEGDA polymer are individually passed through a 20 mL column of activated alumina basic (approximately 10 mL of activated basic) to remove any polymerization inhibiting storage agents such as monomethyl ether of hydroquinone (MEHQ) or butylated hydroxytoluene (BHT). The preparation of the polymer solutions separately promotes improved solubility of these macromolecules in the aqueous buffer, which is desirable for creating optimal reaction conditions when the two solutions are subsequently mixed. The ETTMP 1300 solution are prepared at a concentration of 40 weight percent polymer (i.e., 1.725 mL of buffer for every 1 mL of ETTMP 1300 polymer). The 40 weight percent ETTMP 1300 solution is preferred for fabricating the hydrogel as it is described here. Lower ETTMP 1300 weight fractions may demonstrate greater insolubility and hence thermodynamically limit gelation, while higher ETTMP 1300 polymer fractions may have larger kinetic restrictions leading to higher than desirable sol fraction in the formed hydrogel system. The PEGDA solution of this example is prepared with a concentration that ensures that two conditions are met: (i) the overall polymer fraction of the mixture of the PEGDA and ETTMP 1300 solutions totals 15%; and (ii) the PEGDA solution contains a sufficient fraction of PEGDA such that the stoichiometry of the acrylate and thiol functional groups is equal. For example, a 8.456 weight percent solution of PEGDA (polymer with average Mₙ=686 g/mol) could be used in a method of treating a peripheral nerve or a method of sealing tissue. The absolute weight percent of PEGDA used may change depending on the average molecular weight of the PEGDA polymer. In this example, the average molecular weight of the PEGDA polymer can vary between 675 and 725 g/mol.

Once the individual polymer solutions have been prepared, if they are stored they can be stored under 4° C. conditions and away from light sources in order to prevent free radical homopolymerization of the PEGDA. To prepare the hydrogel, a known volume of the ETTMP 1300 solution should be aliquoted into a clean tube/container followed by a stoichiometrically equivalent amount of the PEGDA solution. Based on a PEGDA molecular weight of 686 g/mol the volume ratio of the two solutions is 1:4 (volume of 40 wt % ETTMP 1300 solution: volume of 8.456 wt % PEGDA solution). Once the PEGDA solution is combined with the ETTMP 1300 solution the mixture can be briefly vortexed before being drawn up into a syringe or other applicator. The mixed solution will appear cloudy at first but become progressively clearer as gelation proceeds. Alternatively, the two polymer solutions can be delivered using a double barreled syringe where the diameters of the syringes can be selected to achieve the specific volumetric mixing ratios (including but not limited to 1:1, 1:2, 1:4, 1:8, 1:10) defined by final gel formulation (wt %). If necessary the two solutions can be combined in a mixing chamber and dispensed through an appropriate tip (spray, sheet, stream delivery). The final viscoelastic hydrogel that is formed at the completion of the reaction is transparent.

Prior to reaching the critical gelation point the polymer sol solution behaves as a viscous fluid and is hence readily injected or applied to the peripheral nerve lesion. The solution becomes progressively more viscous as the Michael addition reaction proceeds and larger molecular weight polymer species are formed from the numerous nucleation sites within the solution. The time until gelation is reached following mixing of the individual polymer solutions is dependent on the pH of the aqueous buffer, which in turn is dependent on the temperature of the solution. The more basic the pH, the faster is the time to gelation. For hydrogels prepared in isotonic phosphate buffer saline with pH=7.2 at room temperature, the time to gelation occurs over approximately 8-10 minutes.

Once formed, the PEG based hydrogel of this example displays a characteristic syneresis (shrinking) phenomena. Unlike many other hydrogels developed previously the PEG based hydrogel of this example will not swell uncontrollably following application to the peripheral nerve lesion. The 15 wt % hydrogel currently described will shrink by approximately 25% of its original weight, expelling the excess buffered saline into the surrounding environment. The hydrogel platform also has a modulus of elasticity similar to peripheral nerve tissue (˜0.1-0.2 MPa) and possesses resilience on handling that was favored by neurosurgeons during preliminary evaluation. This current hydrogel system is biodegradable and degrades in vitro when incubated in PBS at a rate that suggests total degradation over a 6 month period. The hydrogel has also demonstrated an ability to controllably release the small molecule corticosteroid methylprednisolone with first order kinetics over a period of several weeks in vitro.

The use of the PEG based hydrogel of this example may facilitate the controlled long term delivery of steroids, anesthetics and other analgesic compounds for the treatment of peripheral nerve syndromes caused by degenerative, inflammatory or trauma conditions that involve the direct damage, entrapment or impingement of peripheral nerve roots.

The PEG based hydrogel can be used to deliver:
corticosteroids such as methylprednisolone
anesthetics such as lidocaine, bupivacaine, ropivacaine, and chloroprocaine
analgesics such as morphine, fentanyl, sufentanil, and pethidine
growth factors such as neurotrophin-3 (NT-3) and brain-derived neurotrophic factor (BDNF).

The PEG based hydrogel of this example may be used in a variety of neurosurgical and interventional pain management applications, including but not limited to:
As part of an epidural steroid injection to mitigate pain, inflammation, or to reduce epidural fibrosis on the spinal dural sac and exiting neural elements;
As part of the procedure known as a selective nerve root block;
As part of the procedure known as a caudal injection;
As part of the procedure known as a facet block;
As part of the procedure known as a sacroiliac injection or block;
Treatment of carpal tunnel syndrome;
Treatment of lateral epicondylitis (Tennis elbow);
As a dural sealant for the spine, spinal cord, nerve roots, and surrounding structures;
As a dural sealant for cranial applications;
To deliver drugs, cells, and other substances to the spinal coverings, neural elements, and surrounding structures;
To deliver drugs, cells, and other substances to the intervertebral disc, disc annulus, end plates, and surrounding structures; and
To deliver drugs, cells, and other substances to the vertebral bones and articulating processes including the vertebral body, lamina, facet joint, joint capsule, pars interarticularis, neural foramen, transverse process, spinous process, and surrounding ligaments and other supporting structures.

The references cited throughout this application are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawing.

What is claimed is:

1. A method of treating a peripheral nerve comprising: administering a PEG based hydrogel to patient in need thereof, the PEG based hydrogel comprising an overall polymer weight concentration of less than or equal to 50% at the time of curing, wherein administering includes applying the PEG based hydrogel to a site at or near the peripheral nerve.

2. The method of claim 1, wherein applying the PEG based hydrogel includes applying a composition including precursors of the PEG based hydrogel to the site.

3. The method of claim 2, wherein applying the composition includes in injecting the composition at the site.

4. The method of claim 3, wherein the composition includes an aqueous buffer or solution.

5. The method of claim 4, wherein the composition includes at least on additional agent selected from the group consisting of one of one or more therapeutic agent, a biological epitope, and a specific crosslinker.

6. The method of claim 4, wherein the PEG based hydrogel is formed via a step growth, base-catalyzed reaction between a donor and an acceptor, the donor having a nucleophilic functional group and the acceptor having an electrophilic functional group, wherein the precursors include the donor and the acceptor.

7. The method of claim 6, wherein the nucleophilic functional group is a thiol and the electrophilic functional group is an acrylate and together the thiol and the acrylate form a thioether.

8. The method of claim 6, wherein the donor is a trifunctional thiol polymer and the acceptor is a bifunctional acrylate polymer.

9. The method of claim 6, wherein the donor is ethoxylated trimethylolpropane tri-3-mercaptopropionate and the acceptor is poly(ethylene glycol)diacrylate.

10. The method of claim 6, wherein the PEG based hydrogel comprises at least one type of hydrolytically labile functional group selected from the group consisting of esters, amides, anhydrides, epoxides, carbamates and ureas.

11. The method of claim 4, wherein the composition includes a biological epitope selected from the group consisting of a peptide, a protein, an antibody and an aptamer.

12. The method of claim 11, wherein the biological epitope is selected from the group consisting of RGD and IKVAV.

13. The method of claim 4, wherein the PEG based hydrogel includes enzymatically labile functional groups or substrates.

14. The method of claim 4, wherein the composition further comprises at least one therapeutic agent.

15. The method of claim 14, wherein the at least one therapeutic agent has a concentration of 0.01 mg to 120 mg in 0.1 ml to 20 ml of the PEG based hydrogel.

16. The method of claim 14, wherein the at least one therapeutic agent is selected from the group consisting of an anti-inflammatory drug, a steroid, a surgical analgesia, an enzyme, and a growth factor.

17. The method of claim 14, wherein the at least one therapeutic agent includes at least one of corticosteroid, methylprednisolone, an anesthetic, lidocaine, bupivacaine, ropivacaine, chloroprocaine, an analgesic, morphine, fentanyl, sufentanil, pethidine, an enzyme, chrondrotinase ABC, a growth factor, neurotrophin-3, nerve growth factor, or brain-derive neurotrophic factor.

18. The method of claim 4, wherein administering includes delivering 0.1 ml to 20 ml of the PEG based hydrogel.

19. The method of claim 4, wherein administering includes delivering the PEG based hydrogel as at least part of one of the group consisting of an epidural steroid injection, a selective nerve root block procedure, a caudal injection procedure, a facet block procedure, a sacroiliac injection or block procedure, a treatment of carpal tunnel syndrome, a treatment of lateral epicondylitis, a dural sealant, a substance delivery system, and treating an articulating process.

20. The method of claim 4, wherein administering includes delivering the PEG based hydrogel by a cervical interlaminar injection, a thoracic interlaminar injection, a lumbar interlaminar injection, a lumbar transforaminal injection and a lumbar caudal injection.

21. A composition comprising a PEG based hydrogel with an overall polymer weight concentration of less than or equal to 50% at the time of curing.

22. The composition of claim 21, wherein the PEG based hydrogel is formed via a step growth, base-catalyzed reaction between a donor and an acceptor, the donor having a nucleophilic functional group and the acceptor having an electrophilic functional group.

23. The composition of claim 22, wherein the PEG based hydrogel is formed between two poly(ethylene glycol) (PEG) macromers where one of the macromers has a degree of branching equal to or greater than 3 while the other PEG macromer has a degree of branching equal to or greater than 2.

24. The composition of claim 22, wherein the donor is a trifunctional thiol polymer and the acceptor is a bifunctional polymer.

25. The composition of claim 22, wherein the donor is ethoxylated trimethylolpropane tri-3-mercaptopropionate and the acceptor is poly(ethylene glycol)diacrylate.

26. The composition of claim 22, wherein the PEG based hydrogel comprises at least one type of hydrolytically labile functional group selected from the group consisting of esters, amides, anhydrides, epoxides, carbamates and ureas.

27. The composition of claim 22, wherein PEG based hydrogel includes a biological epitope is selected from the group consisting of peptides, proteins, antibodies and aptamers.

28. The composition of claim 27, wherein the biological epitope is selected from the group consisting of RGD and IKVAV.

29. The composition of claim 22, wherein the PEG based hydrogel includes enzymatically labile functional groups or substrates.

30. The composition of claim 22, wherein the PEG based hydrogel further comprises at least one therapeutic agent.

31. The composition of claim 30, wherein the at least one therapeutic agent has a concentration of 0.01 mg to 120 mg in 0.1 ml to 20 ml of crosslinked hydrogel.

32. The composition of claim 30, wherein the at least one therapeutic agent is selected from the group consisting of an anti-inflammatory drug, a surgical analgesia, an enzyme and a growth factor.

33. The composition of claim 30, wherein the at least one therapeutic agent includes at least one of corticosteroid, methylprednisolone, an anesthetic, lidocaine, bupivacaine, ropivacaine, chloroprocaine, an analgesic, morphine, fentanyl, sufentanil, pethidine, an enzyme, chrondrotinase ABC, a growth factor, neurotrophin-3, nerve growth factor, or brain-derive neurotrophic factor.

34. A method of sealing tissue comprising:
mixing a first component and a second component to form a crosslinked hydrogel, the first component having a degree of functionality greater than or equal to three and selected from the group consisting of a branched monomer, a multifunctional monomer, a branched polymer and a multifunctional polymer; the second component having a degree of functionality greater than or equal to two and selected from the group consisting of a branched monomer, a multifunctional monomer, a branched polymer and a multifunctional polymer; and
administering the first component and second component to tissue in situ prior to or during the step of mixing.

35. The method of claim 34, wherein one of the first component or the second component contains electrophilic functional groups and the other of the first component or the second component contains nucleophilic functional groups.

36. The method of claim 35, wherein mixing includes adding the first component and the second component in stoichiometric equivalencies relative to functional groups.

37. The method of claim 34, wherein mixing includes adding the first component and the second component in a buffering medium.

38. The method of claim 37, wherein the buffering medium has a pH of greater than seven.

39. The method of claim 37, wherein the buffering medium has a pH of 8.5 to 9.5.

40. The method of claim 34, wherein the first component is PEG-diacrylate and the second component is ethoxylated-trimethylolpropan tri(3-mercaptopropionate).

41. The method of claim 40, wherein mixing includes adding PEG-diacrylate and ethoxylated-trimethylolpropan tri(3-mercaptopropionate) in stoichiometric equivalence relative to acrylate and thiol concentrations.

42. The method of claim 34, wherein the crosslinked hydrogel has a polymer weight percent of 10 to 30 percent.

43. The method of claim 34 further comprising incorporating at least one biological epitope in the crosslinked hydrogel.

44. The method of claim 43, wherein the at least one biological epitope includes at least one peptide.

45. The method of claim 44, wherein the at least one peptide includes at least one of RGD, IKVAV or YIGSR.

46. The method of claim 44, wherein incorporating includes conjugating one or more of the at least one peptide to the crosslinked hydrogel by reacting a sulfhydryl group thereon with a functional group on the crosslinked hydrogel.

47. The method of claim 44, wherein incorporating includes modifying the peptide with vinyl functionality including acrylates using conjugation techniques.

48. The method of claim 34 further comprising incorporating a crosslinker in the crosslinked hydrogel.

49. The method of claim 48, wherein the crosslinker is succinimidyl-([N-maleimidopropionamido]-ethyleneglycol) ester.

50. The method claim 34, wherein tissue is dural tissue damaged in an accident or surgery.

51. The method of claim 52, wherein, the dural tissue is cranial.

52. The method of claim 51, wherein the dural tissue is spinal.
